(19) United States
(12) Patent Application Publication
(10) Pub. No.: US 2009/0061473 A1
(43) Pub. Date: Mar. 5, 2009

(54) Measurement of Carbonaceous Particles in Biological Samples

(76) Inventors: Rajiv Krishna Saxena, Research Triangle Park, NC (US); M. Ian Gilmour, Research Triangle Park, NC (US); Michael D. Hays, Research Triangle Park, NC (US)

Correspondence Address: SUMMA, ADDITON & ASHE, P.A., 11610 NORTH COMMUNITY HOUSE ROAD, SUITE 200, CHARLOTTE, NC, 28277, US

(21) Appl. No.: 11/846,587

(22) Filed: Aug. 29, 2007

(51) Int. Cl. C12Q 1/02

(52) U.S. Cl. 435/29; 435/40.5

(57) ABSTRACT

Disclosed is a method of quantitative estimation of carbonaceous particles in biological samples such as biological cells and tissues.

### FIELD OF THE INVENTION

The invention relates to methods of measuring carbonaceous particles (i.e. particles containing at least some carbon in elemental form) present in biological samples (e.g., cells or tissues).

### BACKGROUND

The risks to human health from airborne particles, such as those in diesel exhaust, have been of great concern worldwide.

Fine particle matter in diesel exhaust is known to exert significant toxicological effects in human and animal lungs.

Carbonaceous particles such as diesel exhaust particles (DEP) are a significant component of urban air and have been shown to cause pulmonary inflammation in experimental animals (Ma and Ma, 2002, Singh et al, 2004, Inoue et al, 2005). Exposure to DEP induces enhanced responsiveness to allergens and a weakened immune responsiveness to bacterial infections (Saxena et al, 2003a, Yin et al, 2003, Dong et al, 2005, Gilmour et al 2006).

The potential impact that DEPs may have on human health, has in recent years boosted the scientific research agenda directed at understanding the relationship between exposure to DEP and human health.

The effects of carbonaceous particles and the retention of diesel exhaust particles in biological cells and tissues have not been effectively addressed. This is because; a sensitive quantitative technique for the accurate and objective assessment of accumulation of diesel exhaust particles (DEP) inside lung cells and tissues is not yet available.

Several methods for the measurement of accumulation of diesel exhaust are known in the art. For instance, U.S. Pat. No. 5,571,945 discloses an apparatus for measuring the amount of particulate matter in a gas, such as for environmental sampling.

U.S. Pat. Nos. 5,110,747 and 5,196,170 disclose methods of measurement of carbon particle concentration of diesel exhaust by collecting the particulates on a high efficiency filter while measuring the amount of sampled gas passing through the filter.

The methods of measuring diesel exhaust disclosed in the aforementioned patents are essentially directed at measuring the elemental carbon in airborne diesel exhaust particles and not for the quantitative estimation of particles containing elemental carbon, present in biological samples.

Accordingly, there is a need for efficient, inexpensive, and accurate techniques of estimation of carbonaceous particles in biological samples (e.g., tissue and cells).

### SUMMARY OF THE INVENTION

In one aspect, the invention provides a method of estimating carbonaceous particle taken up by cells like epithelial cells and macrophages in tissue culture. This will assist in the study of particle—cell interactions and linking toxicity to the dose of internalized carbonaceous particles.

In another aspect, the invention provides a method of estimating carbonaceous particles deposited in lung tissue as a result of exposure to airborne carbonaceous particles e.g. diesel exhaust particles or occupational exposure to different types of carbon dust (e.g. in coal miners).

In yet another aspect, the invention provides a method of estimating carbonaceous particle present in biological samples (e.g., cells, tissues) where the biological samples are solubelized leaving behind insoluble carbonaceous particles that can be separated and analyzed for the amounts of elemental and organic carbon they contain.

In yet another aspect, the present invention embraces a method of estimating carbonaceous particle take-up in a biological sample where the biological sample is first dissolved using a solubilizing agent and the residual insoluble carbonaceous particles are separated and analyzed to distinguish between elemental and organic carbon contents.

In yet another aspect, the present invention embraces a method of estimating carbonaceous particle deposited in lung tissue where the lung tissue is dissolved in biological detergent, sodium dodacyl sulfate or commercial tissue solubilizer like SOLVABLE® (Perkin Elmer Life and analytical Sciences, Ontario, Canada), and the carbonaceous particles that remain insoluble are then separated from the dissolved lung tissue and analyzed for the estimations of elemental and organic carbon contents.

In yet another aspect, the present invention embraces a method of estimating carbonaceous particle either taken up by cells or deposited in lung tissue where the biological sample is solubilized and insoluble carbonaceous particles are isolated by using a high speed centrifuge from the liquefied biological sample and analyzed to distinguish between elemental and organic carbon contents.

The foregoing, as well as other objectives and advantages of the invention, and the manner in which the same are accomplished, are further specified within the following detailed description and its accompanying drawings.

### BRIEF DESCRIPTION OF THE DRAWINGS

FIG. 1 illustrates the carbon analyzer-produced thermograms for ultrafine carbon black (i.e., UFCB, panel A), Diesel exhaust particles (DEP) (panel B), LA4 lung epithelial cells (panel C), and sodium dodecyl sulfate (panel D)

FIG. 2 illustrates the organic carbon and elemental carbon analysis of mixtures of LA4 lung epithelial cells and DEP.

FIG. 3 illustrates the organic carbon and elemental carbon analysis of DEP-exposed LA4 lung epithelial cell samples with and without the spiking of a standard DEP solution.

FIG. 4 illustrates the kinetics and dose dependence of DEP uptake by LA4 lung epithelial cells in culture.

FIG. 5 illustrates the comparison of uptake of DEP and UFCB by LA4 lung epithelial cells and MHS alveolar macrophage cells.

FIG. 6 illustrates the estimation of uptake of nano-diamonds by lung epithelial cells in culture.

FIG. 7 illustrates the estimation of retention and time kinetics of dissipation of DEP from mouse lung tissue.

### DETAILED DESCRIPTION

The present invention is a method of measuring carbonaceous particles (i.e. particles with some proportion of carbon in elemental form, e.g., diesel exhaust particles (DEP), ultra-fine carbon black (UFCB), nano-diamonds and carbon nanotubes) in biological samples (e.g., cells or tissues).

Those having ordinary skill in the art will know that carbon is not present in its elemental form in biological cells and tissues. Most constituents that make up cells and tissues are carbon compounds in which carbon is present in combination with other elements like hydrogen, oxygen, and sulfur. This form of carbon is called organic carbon (OC) and accounts for 100 percent of the carbon present in normal biological cells and tissues. Many environmental pollutants like DEP and cigarette smoke (to a lesser extent) contain carbon in elemental form (i.e., elemental carbon or EC). Such pollutants can be deposited in lungs and possibly in other tissues. Nano-diamonds and carbon nanotubes (almost 100 percent elemental carbon) are not environmental pollutants but occupational exposure to these agents may occur.

The mechanism of DEP induced changes in lungs remains unclear. DEP comprise a central carbonaceous core, which adsorb a large number of organic components. Different fractions of DEP may exert different biological effects (Ma and Ma, 2002, Saxena et al 2003b, Siegel et al, 2004). The fate and transport of DEP, once they reach the lung alveoli, is also not clear. Alveolar macrophages may engulf DEP and move away from the alveolar spaces through the mucociliary escalator. Inhaled DEP may also come in close contact with the epithelial cells that line all airways as well as the alveoli. Whether a portion of DEP is ingested by the lung epithelial cells and how long it is retained in these cells is not known. The issue of retention of DEP in lungs may have many important implications. DEP associated organic compounds may continue to leach from DEP retained in lungs. Moreover, long-term presence of DEP in epithelial cells may influence their survival, responsiveness, and susceptibility to airborne pathogens and other environmental and physiological stimuli.

Thus far, there exists no accurate method to assess exposure of biological tissues or cells to such agents e.g., carbonaceous particles.

The present method is useful in assessing deposits of carbonaceous particles (e.g., DEP, carbon nano-tubes, nano-diamonds, and ultra-fine carbon dust) that may be deposited in cells and tissues because of environmental or occupational exposures. For example, there is no data available on how much carbon dust gets deposited in-lungs of coal miners, or how much diesel exhaust particles are deposited in the lungs of traffic policemen, or how much DEP is present in the lungs of people living in different areas of the country with different levels of airborne pollutants. The present method enables compilation of such data necessary for assessing the exposure of human beings and animals to pollutants containing elemental carbon. Further, there may also be research usage of this technique for the study of mechanisms of uptake of carbon particles by cells and its biological regulation.

The method according to the present invention enables estimation of the amount of elemental carbon in cells and tissues. More particularly, the present invention is a method for the direct assessment of carbonaceous particles uptake by biological cells and tissues. In particular, the present invention is a method for the direct assessment of carbonaceous particles, uptake by lung epithelial cells, and alveolar macrophages in tissue culture, or of deposits of carbonaceous particles in lungs.

This is accomplished by solubilizing all the organic components in the biological sample and isolating elemental carbon that is insoluble. This isolated elemental carbon, with some impurity of organic carbon, is analyzed by using a carbon analyzer (e.g., thermo-optical carbon analyzer).

The biological sample used in the present invention could be cells and tissues of any organism (e.g., human and rodents). In the examples provided, these biological samples are either lung cells (e.g., lung epithelial cells or alveolar macrophages) or samples of lung tissue with prior exposure to carbonaceous particles.

The present invention discloses a simple method capable of quantitative measurement of the uptake of carbonaceous particle mass by cells cultured in vitro (i.e., outside the body in culture flasks where live and functional cells can be kept and grown in tissue culture media). The present method takes advantage of a carbon analyzer commonly employed to determine microgram quantities of organic and elemental carbon in samples of atmospheric and combustion source aerosols.

Because carbon constituents of cells and tissues are organic in nature (proteins, nucleic acids, lipids, etc.) and standard DEP preparations (National Institute of Standards and Technology) comprise predominantly elemental carbon (90 percent w/w), we could use the carbon analyzer to directly estimate the amount of DEP in cells that had ingested DEP. Analysis of cells alone, however, showed that the organic constituents of the cells interfered with the direct estimation of elemental carbon present in the ingested DEP. To overcome this problem several important sample pre-treatment steps were developed. These steps helped to reduce the interfering constituents in the sample. Once these steps were established, the new technique could precisely estimate DEP and ultra-fine carbon black (UFCB) particle uptake by cultured lung epithelial cells (i.e., LA4 lung epithelial cells) and alveolar macrophage cells. The results of this experiment showed for the first time that this uptake is dose and time dependent, and that the macrophages cells engulf DEP and UFCB with comparable efficiencies, whereas LA4 epithelial cells ingest substantially more DEP than UFCB.

The present method of measuring carbonaceous particle uptake by a biological sample, where the biological sample was exposed to carbonaceous particles, can be better understood by the following example:

Biological sample i.e., LA4 murine lung epithelial cell and MHS murine alveolar macrophage cell lines were obtained from American Type Cell Culture.

This biological sample was maintained in RPMI1640 culture medium supplemented with glutamine (2 mM), HEPES buffer (25 nM), gentamycin (20 μg/ml), and fetal bovine serum (FBS, 10 percent V/V). Diesel exhaust particles (DEP Standard Reference Material 2975) were purchased from the National Institute of Standards and Technology (NIST), Gaithersburg, Md. Lympholyte-M, a Ficoll® suspension used for isolating viable mouse cells by density gradient centrifugation was obtained (e.g., from Accurate Chemical and Scientific Corp. Westbury, N.Y.).

A DEP stock suspension (5 mg/ml) was prepared in sterile normal saline and sonicated for one minute using a probe sonicator at maximum amplitude (Microson Ultrasonic Cell Disrupter, Farmingdale, N.Y.). Cells were seeded in a culture at a cell density of 2×10⁴/ml in six well culture plates and after 72 hours, the sonicated DEP suspension was added at desired concentrations. Control and DEP treated cells were harvested after desired intervals of time by trypsinization (Trypsin-EDTA 1X from Gibco; 2 min), and collected by centrifugation at 800 rpm for 10 min. Those skilled in the ordinary art would know that in a cell culture, cells frequently adhere to the plastic surface of the culture vessel. Trypsin, an enzyme commonly found in the digestive tract, can be used to detach cells from each other and from the culture vessel and therefore allow better observation and experimentation to be conducted. This process is called trypsinization.

Cell pellets were resuspended in 5 ml RPMI culture medium containing 1 percent fetal bovine serum (FBS), layered on top of 2 ml Ficoll solution (Lympholyte-M), and centrifuged at room temperature (1600 rpm×20 min). Cells with ingested DEP formed a black band at Ficoll-RPMI interface whereas free DEP settled at the bottom of the Ficoll layer. Cells harvested from the interface were washed twice with RPMI medium containing 1 percent v/v FBS (centrifugation at 800 RPM for 10 minutes in a benchtop centrifuge) and counted in a hemocytometer (i.e., an instrument used to calculate the concentration of cells in a specific volume of a fluid).

An amount of 0.5 ml of 2 percent SDS solution (Sodium dodecyl sulfate, 2 percent W/V in normal saline), was added to 0.5 ml of Ficoll purified cell suspension and the mixture vortexed immediately. Cells in SDS were kept in boiling water for 15 minutes with intermittent vortexing to completely solubilize the cellular constituents. Ingested DEP remained insoluble in SDS and was isolated by high-speed centrifugation (Eppendorf microfuge, 13,200 rpm×20 min). In order to increase the bulk of the DEP pellet and prevent loss of DEP during washing steps, 100 μl of a 5 mg/ml suspension of silica powder (Sigma Aldrich, St. Louis, Mo.) in normal saline was added to each tube at the same time when SDS was added. DEP/silica pellets were washed once with 1 ml of hot 1 percent SDS and twice with 1 ml normal saline (a solution of 0.89 gram sodium chloride in 100 milliliters water). Washed pellets were suspended in 50 μl normal saline and transferred to 1.5 cm² quartz filter punches. Filters were dried overnight in in-vacuo at ˜50° C. and analyzed for elemental and organic carbon using a carbon analyzer.

The organic and elemental carbon (OC and EC) in the DEP-silica pellets were measured by using a thermal-optical carbon analyzer (Sunset Laboratories; Forest Grove, Oreg.), and utilizing the National Institute for Occupational Safety and Health Method 5040 for estimating carbon in diesel particulate matter (18). Exhaustive details of the method have been provided elsewhere (2,4). Briefly, vacuum-dried DEP-silica samples on quartz filters were heated to ˜850° C. stepwise in a temperature-programmable oven under helium atmosphere (He phase). Organic matter volatilized under these conditions were oxidized to CO₂ in the presence of MnO₂ catalyst, and then reduced to methane. The methane generated was detected and quantified by a flame ionization detector (FID) and the organic carbon (OC) present in the sample could be computed from the amount of methane evolved during this step. Next, after a brief cooling respite, the samples were again heated stepwise up to 900° C. in a Helium-Oxygen atmosphere whereby the non-volatile elemental carbon was oxidized to CO2. CO₂ was then converted to methane that was quantified as above. Elemental carbon could be assessed from the amount of methane evolved during the Helium/Oxygen phase.

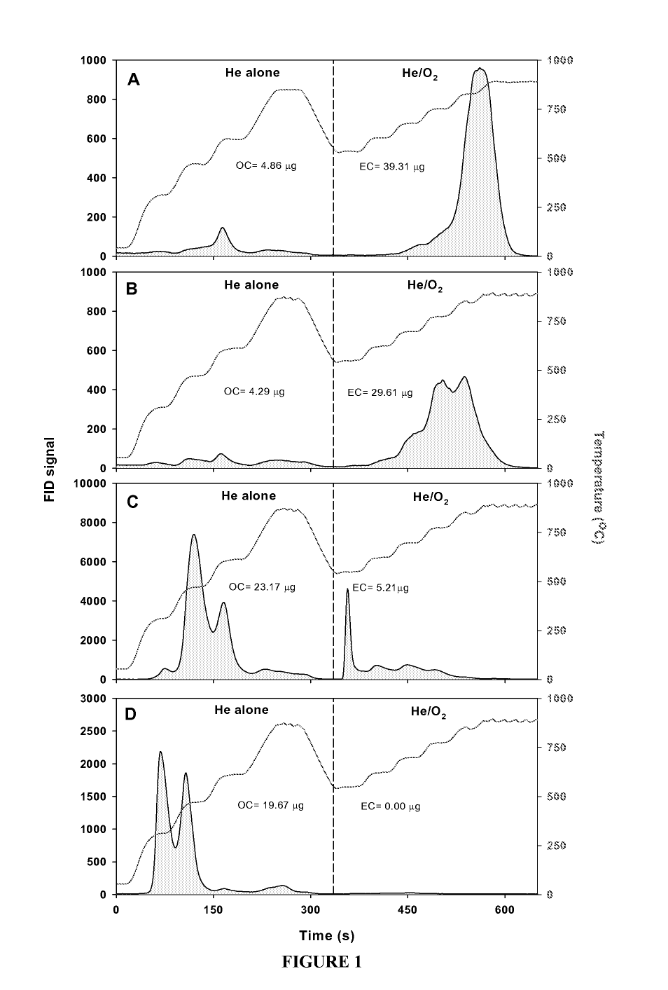

For an accurate measurement of OC and EC concentrations of DEP in microgram quantities in UFCB and LA4 cells, a thermo-optical carbon analyzer is used. FIG. 1 panels A and B show the carbon analyzer-produced thermograms for ultrafine carbon black (UFCB, panel A), and the DEP (panel B). To obtain these results Quartz filters (1.45 cm²) were loaded with the pre-treated biological sample material and dried overnight at 50° C. in a vacuum oven. Material on the filter was subjected to OC-EC analysis i.e., panel A—UFCB (45 μg), panel B—DEP (30 μg), panel C—LA4 cells (1.5×10⁵ cells), panel D—sodium dodecyl sulfate (20 μg). The y-axis in FIG. 1 refers to the flame ionization detector (FID) signal as a measure of methane generated. These results indicate that the UFCB and DEP samples are predominantly EC (89 and 87 percent, respectively).

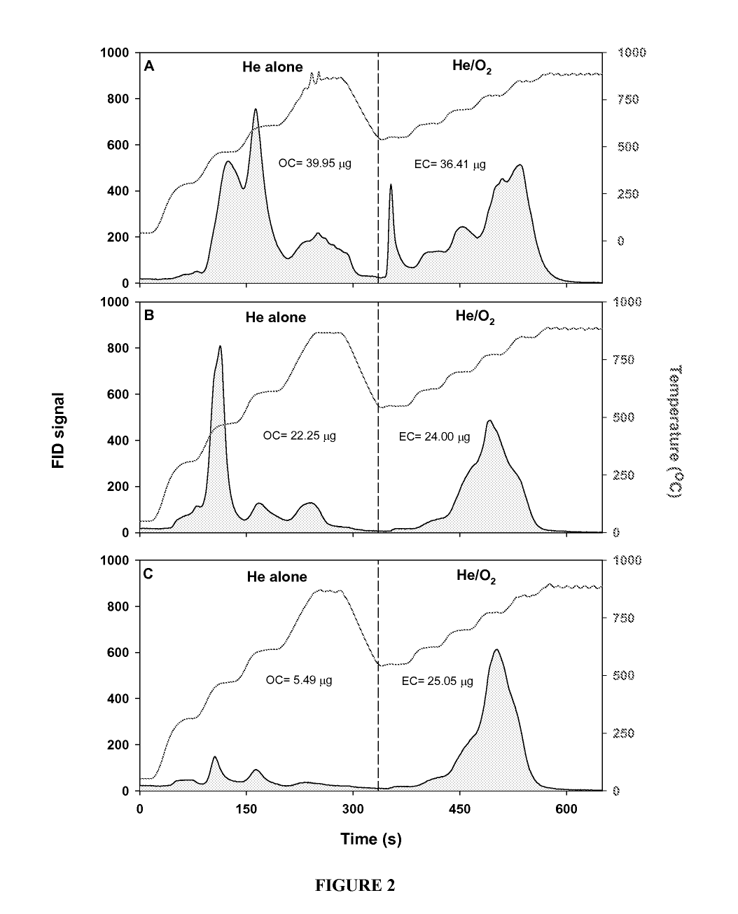

Carbon analysis results of the control LA4 cell preparation given in FIG. 2 (panel C) indicate that a significant portion of the cell associated carbon evolved in the He—O₂ phase due to partial charring (pyrolysis) of the cellular material that generated fresh elemental carbon from the organic carbon present in the cells.

For the method to succeed, it is necessary to first isolate the cells that have ingested DEP from any loosely adhering and free-floating extra-cellular DEP in the culture medium. This objective is achieved by detaching the DEP exposed epithelial cells by trypsinization followed by separation of cells from free DEP by Ficoll density gradient centrifugation. The total analyzer-measured OC in these purified cell samples is expected to be predominantly of cellular origin with only a small contribution from the organic compounds adsorbed on DEP. Whereas, the EC in these samples is expected to come only from DEP (since normal cells do not typically contain any EC). On this presumption, a carbon analyzer is used to determine the OC and EC values in DEP-exposed LA4 epithelial cell samples. This approach was, however, found to be problematic because during the initial helium phase, a portion of cellular organic carbon got pyrolized (charred) to black elemental carbon form, and thus interferes with the accurate estimation of EC present in DEP.

From the results illustrated by the above-referenced FIG. 2, it is evident that certain classes of cellular organic matter, especially large polymeric entities, may not be volatilized during the initial helium phase and may instead be thermally degraded to elemental carbon. To overcome this challenge of airborne carbonaceous particle samples, a laser based monitoring system is used to account for OC pyrolysis and factors in pyrolized OC when calculating the OC-EC split point. However, for the biological sample comprising large amounts of cellular components and relatively small amounts of DEP derived EC, it was found that the laser-based system did not give a satisfactory OC-EC split. This was because the pyrolized material interfered with the EC estimate, which was needed to determine the concentration of cell-accumulated DEP.

This pyrolytically formed elemental carbon (EC) overlapped with the DEP EC peaks (compare the He—O₂ phase in panels B and C), complicating the direct estimation of the DEP EC concentration in the presence of cellular matter. To resolve this problem, the cellular components from the biological samples were removed while retaining only the DEP particles that were ingested for carbon analysis. This was accomplished by solubilizing DEP-exposed LA4 cells in a hot SDS solution (final concentration 1 gram SDS in 100 milliliter normal saline). The residual DEP that was present in the cells was isolated by centrifugation (13,200×g for 20 minutes) whereby DEP collected as a tiny pellet in the bottom of the centrifuge tube. The DEP pellet was transferred to a quartz filter and analyzed for its EC and OC contents. This approach could succeed only if the SDS present adhered to DEP in the pellet did not get charred like the cellular components to yield elemental carbon by heating. If organic material adhered to DEP pellet also got charred, it would have simply created the same artifact as observed when the cellular material were not removed prior to EC/OC analysis. The SDS thermogram in FIG. 1 (panel D) demonstrates that the SDS carbon evolved in its entirety during the helium phase and therefore would not interfere with the DEP EC estimation.

In addition, sodium dodecyl sulfate (SDS) and silica were used to further improve estimation of ingested DEP. LA4 epithelial cells containing intracellular DEP particle matter were isolated by centrifugation, and the cellular pellets were treated with hot SDS (1 gram per 100 milliliter normal saline solution).

The SDS solubilized the cellular components, leaving insoluble DEP particles to be isolated by high-speed centrifugation. Solubilized cellular material present in the supernatant was discarded. FIG. 2 carbon thermograms show DEP-exposed LA4 cells (panel A) and the SDS-treated pellet containing largely DEP (panel B). Comparison of panels A and B clearly illuminates the ability of the SDS treatment to accomplish the objective of removing the cellular component from the test samples. The significant quantity of OC in FIG. 2 panel B thermogram is due to the residual SDS associated with the DEP pellets following the SDS treatment. Further attempts to rinse the SODS from the treated DEP pellet using normal saline gave variable results. The problem was that the microgram quantities of hydrophobic DEP dispersed unevenly in normal saline and tended to stick to the centrifuge tube walls. This problem was solved by adding carrier silica particles along with SDS. Addition of silica particles (500 μg/tube) forced the DEP to settle and resulted in quantitative recoveries of DEP during normal saline washing steps. Silica itself being devoid of carbon caused no interference with the subsequent EC/OC analysis of the samples.

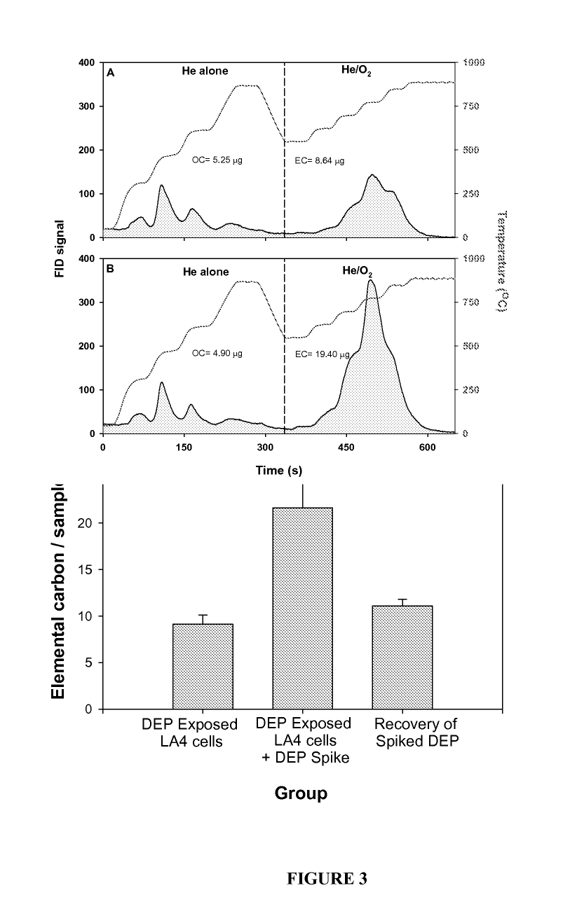

This method's reliability for estimating EC in cellular biological samples was tested by spiking a known amount of DEP into a fully pre-treated solution of DEP-exposed LA4 cells and then estimating the recovery of the spiked DEP. Representative experimental results are exhibited in FIG. 3, which illustrates the OC-EC analysis of DEP-exposed LA4 cell samples with and without the spiking of a standard DEP solution. LA4 cells were cultured with DEP for 18 hours, following which cells were isolated and purified as described in the description of FIG. 1. Purified cells were divided into equal aliquots and spiked with DEP containing 12 μg EC. Control and spiked aliquots were processed for SDS treatment followed by washing with normal saline as described in the FIG. 3C legend. The OC-EC profile of control and DEP-spiked samples is shown in panels A and B, respectively. Data of EC measurements in control and spiked samples in six such independent pairs of control and spiked samples are summarized in histograms in panel C. Error bars show standard deviation of six replicate estimations.

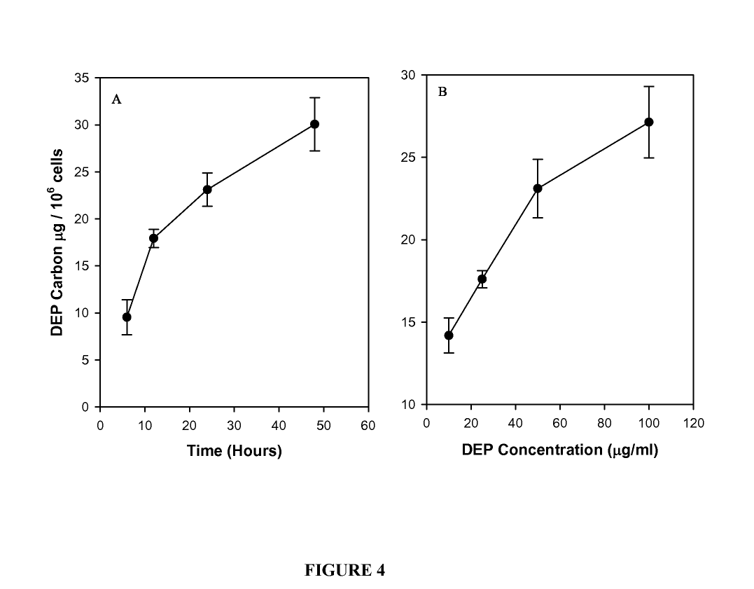

Panel A of the figure shows the carbon analysis results of DEP-exposed LA4 cells, in which case the sample contained 8.64 μg of EC due to cell-ingested DEP. After spiking the identical sample with DEP containing 11.5 μg EC, the total EC measured equaled 19.4 μg, indicating a 94 percent recovery (or 10.76 μg of EC was recovered, see, FIG. 3 panel B). A summary of six replicate experiments shown in FIG. 4C indicate that the recovery of EC due to spiked DEP ranged from 89 to 107 percent (96.5 percent, ±6.2 SD). These results underscore the method's reproducibility for measuring DEP uptake by LA4 cells.

The aforementioned method of measuring carbonaceous particles in a biological sample, which also may be applied to in vivo systems, isolates DEP from a known number of exposed cells and estimates its concentration using a thermo-optical carbon analyzer.

This method has been extensively tested, and has shown high recovery yields and reproducibility when estimating DEP uptake by lung epithelial cells.

This method was also employed to study the kinetics and dose response of DEP uptake by LA4 lung epithelial cells. FIG. 4, Panel A shows LA4 cells cultured in 6 well culture plate in presence of 50 μg/ml of DEP for 6, 12, 24 and 48 hours. Cells were isolated and purified on Ficoll density gradient and subjected to SDS treatment and the pellets of ingested DEP were washed with normal saline. DEP pellets then underwent carbon analysis. Each mean±standard deviation value represents data from four independent culture wells. Panel B shows LA4 cells cultured in 6 well culture plates with 10, 25, 50 and 100 μg/ml DEP for 24 hours and DEP uptake estimated as described above for panel A. Standard deviations of less than 10 percent were observed over the variable incubation periods (10-48 hours) and DEP doses (1-50 μg/ml). Increasing the concentrations of DEP added to the culture medium corresponded to a proportional increase in DEP uptake by the LA4 cells. The kinetics curve (FIG. 4A) shows relatively fast DEP uptake with incubation periods of up to 12 hours tapering thereafter. The amount of DEP taken up by LA4 cells in this experiment ranged from 10 to 30 μg/10⁶ cells.

FIG. 4 panel A shows the kinetics of DEP uptake by LA4 cells. Per million LA4 cells, nearly 10 μg of EC was ingested after a 6 hours incubation period, suggesting significant uptake of DEP by LA4 cells. The uptake increased thereafter until reaching an uptake of 30 μg/10⁶ cells at the maximum 48 hours time point. FIG. 4 panel B shows the effect of DEP concentration in the culture medium on the DEP uptake by LA4 cells over 24 hours. At the highest dose of DEP (100 μg/ml in flasks containing 10 milliliter culture medium and 2×10⁶ cells), DEP uptake was about 25 μg/10⁶ LA4 cells. These method results clearly indicate that DEP uptake by LA4 cells is both time and dose dependent.

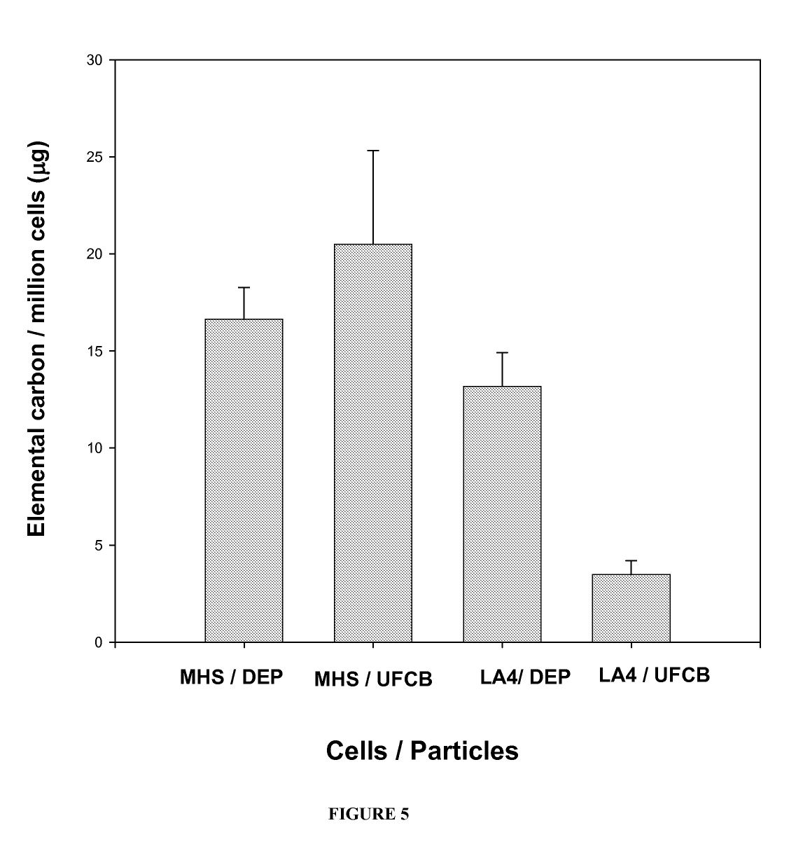

In another aspect, DEP and ultra fine carbon black (UFCB) uptake by LA4 epithelial cells and MHS alveolar macrophages was compaired. FIG. 5 compares the uptake of DEP and UFCB by LA4 epithelial cells and MHS alveolar macrophage cells. LA4 and MHS cells were cultured in 75 cm² culture flasks with or without 100 μg/ml of DEP or UFCB for 6 hours. At the end of the incubation, cells were isolated and uptake of UFCB and DEP per million cells was estimated as described above. Each value is a mean±standard deviation value of data from four replicate culture flasks (* p<0.05). Those of ordinary skill in the art would appreciate that * p<0.05 denotes that the probability of two compared sets of observations not being different is less than 5 percent.

The results as illustrated in FIG. 5 show that on a 10⁶ cell basis, DEP ingestion by LA4 epithelial cells was analogous to that of MHS macrophage cells. In contrast, the consumption of UFCB by epithelial cells was about 3-fold less than that of macrophages. On a per cell basis, the uptake of DEP was comparable in LA4 and MHS cells. While MHS macrophages could ingest both DEP and UFCB equally efficiently, LA4 cells appeared to be significantly more efficient in ingesting DEP than UFCB. These results illustrate the kind of quantitative differences in the ability of different cell types to ingest carbonaceous particles that can be accurately investigated by using the technique described here.

Thus, the present method is a new technique for quantitatively assessing the ingestion of DEP by lung cells. Using this technique, one can measure the time and dose dependent uptake of DEP by LA4 lung cells.

Furthermore, the present method helped to assess the qualitative differences in the uptake of ultrafine carbon black and DEP by LA4 epithelial cells and MHS alveolar macrophages.

The present method may easily be applicable to other carbonaceous particles and will facilitate further work for understanding the fundamental issue of retention of carbonaceous particulates within lungs.

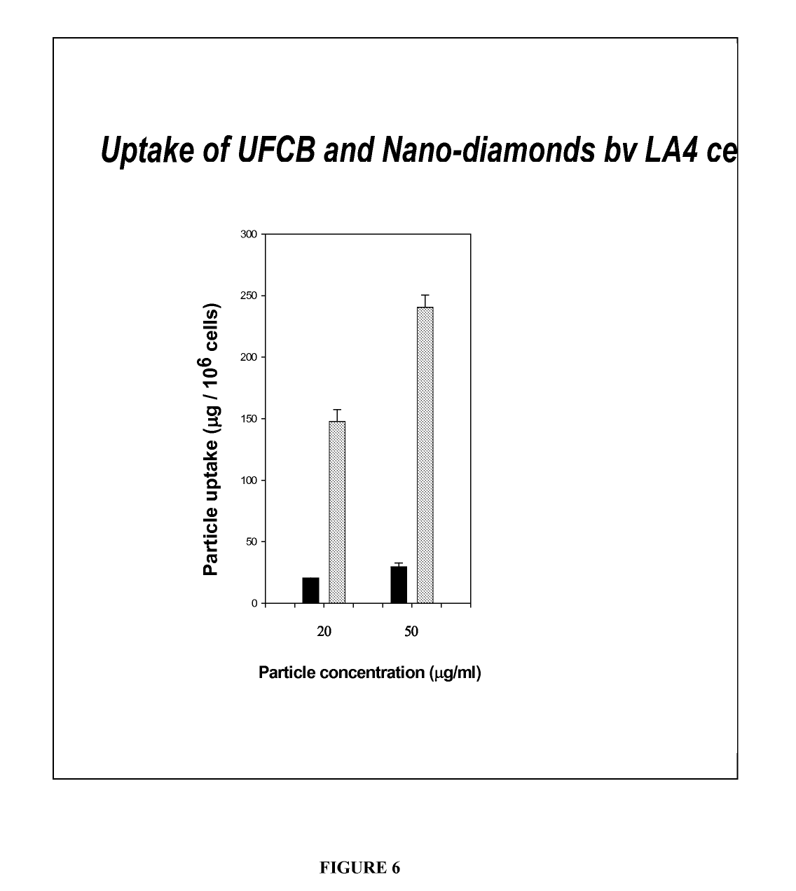

FIG. 6 illustrates the results of an experiment demonstrating the use of the present technique to estimate the uptake of nano-diamonds. In this experiment, LA4 lung epithelial cells were incubated with a suspension of nano-diamonds or ultra-fine carbon black particles (UFCB as control) in tissue culture medium. After 6 hours, LA4 cells were detached by trypsinization and purified by using Ficoll Density gradient centrifugation as described above for the DEP uptake experiments. Cells were solubilized in SDS and insoluble nano-diamonds were collected by high speed centrifugation, and analyzed by using a carbon analyzer. Results in FIG. 6 show that LA4 cells can take up about 10 fold higher amounts of nano-diamonds than ultra-fine carbon black.

The present invention is a method of estimating concentrations of carbonaceous particles in biological cells and tissues (e.g., lung epithelial cells and alveolar macrophages). First, the biological sample that would be evaluated for the carbonaceous particle uptake is measured. For tissue samples, the sample is weighed. For cell samples, the cells in the sample are counted (i.e., by using an instrument such as hernocytometer).

The measured amount of biological sample is then treated such that the elemental carbon present is separated from the sample.

For the accurate measurement of the concentration of carbonaceous particle, the biological tissue sample is treated slightly differently than the biological cell sample.

In case of the biological tissue sample, the moisture of the wet tissue is typically eliminated (e.g., squeezing, using a blotting paper). The tissue is then weighed and preferably cut into small pieces (e.g., 1-2 mm in size).

The amount of tissue sample needed for estimating elemental carbon would typically depends upon the amount of elemental carbon present in the sample. For instance, for estimating the elemental carbon in a coal miner's lungs, a 0.1 gram of tissue sample should be sufficient. Whereas, for estimating the elemental carbon in a cigarette smoker's lungs, a much large tissue sample would be required as the concentration of elemental carbon is relatively low in cigarette smoke.

The tissue pieces are then homogenized in 2 milliliter normal saline (e.g., by using a Polytron homogenizer) until the homogenate is smooth and devoid of any large particles. For solubilizing tissues (like lung tissue), there are two options. For small amounts of fresh lung tissue samples (say up to 100 milligram wet weight), samples may be homogenized in normal saline and then dissolved in 1 percent hot SDS as described for cells. For bigger samples or formalin fixed tissue samples, a commercially available solubilizer (SOLVABLE®), may be used to dissolve the tissues. Tissue pieces are taken in a glass tube and a sufficient amount of SOLVABLE® is added so that all tissue pieces are completely covered with SOLVABLE®. Silica powder 0.5 mg/sample is added as was the case with the cells. Tubes are then kept at 65° C. for 24 hours or longer if the tissue pieces are not completely solubilized. After this step, insoluble carbon particles are isolated by centrifuging the suspension at 80,000×g for 25 minutes. Pellets containing carbon particles are washed twice with normal saline and transferred to quartz filter for carbon analysis. The carbon content is analyzed by using a carbon analyzer (e.g., a thermo-optical carbon analyzer)

Two illustrative experiments using the present technique for estimation of DEP in lung tissues are now described.

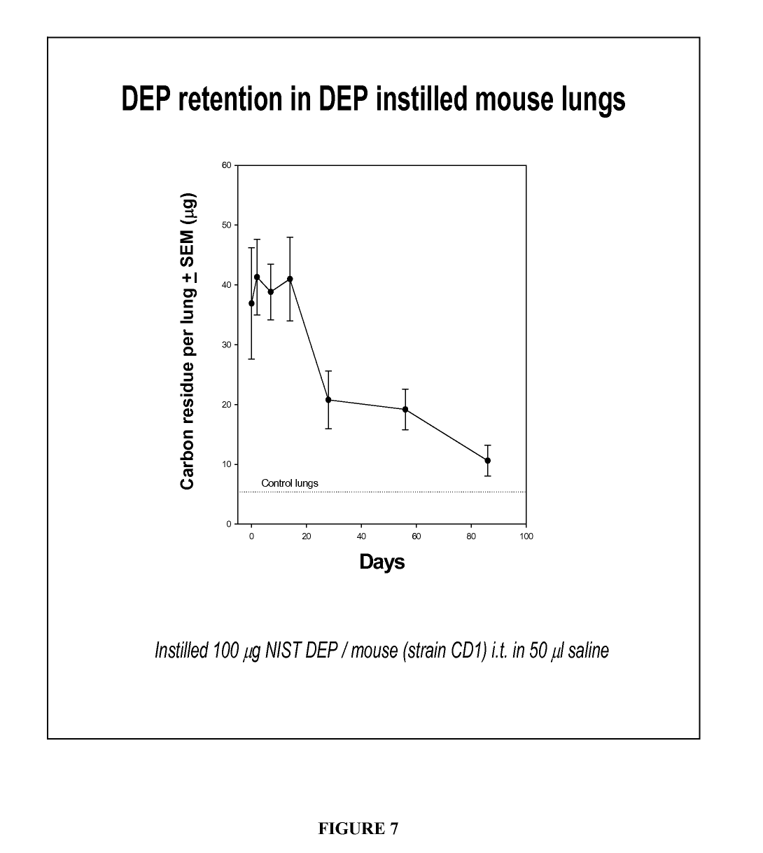

In the first experiment, 100 microgram of DEP (for each mouse) was directly deposited in the lungs of a group of mice by the process of intra-tracheal instillation. At different time points after the DEP instillation, five mice were sacrificed and their lungs were removed. Lungs were homogenized by using a Polytron homogenizer and dissolved in 1 percent hot SDS. Insoluble DEP was isolated by high speed centrifugation (80,000×g for 25 min) and the DEP pellet washed twice with normal saline. Finally the DEP pellets were suspended in 50 micro liters of normal saline and transferred to quartz filters for carbon analysis. Results in FIG. 7 show that the amount of DEP per lung remained more or less static until the end of the second week. After that time point, DEP levels started to fall with time and almost 90 percent DEP was cleared from the lungs by the end of 3rd month.

In the second illustrative experiment, carbon deposited in samples of coal miners' lungs was estimated. Five samples of lung pieces from coal miners and seven samples from control (i.e., no coal miners) lungs were analyzed for the amount of carbon dust present in the samples. Tissues were solubilized in SOLVABLE® and insoluble carbon dust was isolated by high-speed centrifugation, washed, and transferred to quartz filters for carbon estimations. Results are shown as milligram of carbon dust present per gram of the tissue samples in Table 1 (below). (Mean and standard errors have been shown):

TABLE 1
Carbon dust estimation on lungs from coal miners

| | | Carbon deposit |
|---|---|---|
| | Number of | mg EC/g lung total carbon ± Standard |
| Group | samples | Error |
| Control | 7 | 0.44 ± 0.15 |
| Coal miners | 5 | 84.90 ± 36.02 |

In the case of the biological cell sample, the number of cells required for analysis would depend upon the amount of elemental carbon present in them. Preferably, the amount of the biological cell sample should be such that the final load of carbon particles on quartz filter should not be less than 1-micro grams or more than 100-micro grams. More preferably, the final load of carbon particles on quartz filter should not be less than 1-micron grams or more than 50-micro grams

In the specification and figures, typical embodiments and examples of the invention have been disclosed. Unless otherwise noted, specific terms have been used in a generic and descriptive sense and not for purposes of limitation.

1. A method of determining carbonaceous particle concentration in biological cells, comprising the steps of:
Counting the biological cells;
separating elemental carbon, in the biological cells, in the form of washed pellets; and
analyzing the elemental carbon in the pellets.

2. A method according to claim 1, wherein the step of counting biological cells comprises counting the biological cells using a hemocytometer.

3. A method according to claim 1, wherein the step of separating elemental carbon from the biological cells comprises:
solubilizing the biological cells in a solvent; and
isolating, by high speed centrifugation, residual carbonaceous particles in the form of pellets.

4. A method according to claim 3 wherein the step of solubilizing the biological cells in a solvent is preceded by trypsinizing the biological cells to facilitate isolation of biological cells containing carbonaceous particles.

5. A method according to claim 3 wherein the step of solubilizing the biological cells in a solvent comprises dissolving the biological cells in an alkaline solubilizer.

6. A method according to claim 3 wherein the step of solubilizing the biological cells in a solvent comprises dissolving the biological cells in sodium dodecyl sulfate.

7. A method according to claim 3 wherein the step of solubilizing the biological cells in a solvent comprises dissolving the biological cells in a solvent of normal saline, silica, and sodium dodecyl sulfate.

8. A method according to claim 3 wherein the step of isolating residual carbonaceous particles in the form of pellets is followed by the steps comprising:
washing the pellets to remove impurities from the pellets; and drying the washed pellets.

9. A method according to claim 8, wherein the step of washing the pellets comprises:
washing the pellets by suspending the pellets in normal saline to form a mixture;
vortexing the mixture; and
centrifuging the mixture to isolate residual carbonaceous particles in the form of pellets.

10. A method according to claim 8, wherein the step of drying the washed pellets comprises transferring sufficient amount of pellets to a quartz filter such that the total mass of the elemental carbon particles in the pellets on the filter is between about 1 μg and 100 μg.

11. A method according to claim 1, wherein the step of analyzing the elemental carbon comprises measuring the concentration of elemental carbon in the washed pellets using a carbon analyzer.

12. A method of determining carbonaceous particle concentration in biological tissue, comprising the steps of:
weighing a sample of biological tissue;
separating elemental carbon in the form of washed pellets from the biological tissue; and
analyzing the elemental carbon in the pellets.

13. A method according to claim 12, wherein the step of separating elemental carbon from the biological tissue comprises:
homogenizing the cellular component of the biological tissue to form a slurry;
solubilizing the biological tissue in a solvent; and
isolating, by high speed centrifugation, residual carbonaceous particles in the form of pellets.

14. A method according to claim 13, further comprising, prior to the step of homogenizing the cellular component of the biological tissue to form a slurry, dividing the biological tissue into small pieces.

15. A method according to claim 13 wherein the step of solubilizing the biological tissue in a solvent comprises dissolving the biological tissue in an alkaline solubilizer.

16. A method according to claim 13 wherein the step of solubilizing the biological tissue comprises dissolving the biological tissue in sodium dodecyl sulfate.

17. A method according to claim 13 wherein the step of solubilizing the biological tissue comprises dissolving the biological tissue in a solvent of normal saline, silica, and sodium dodecyl sulfate.

18. A method according to claim 13 wherein the step of isolating residual carbonaceous particles in the form of pellets is followed by the steps comprising:
washing the pellets to remove impurities from the pellets; and
drying the washed pellets.

19. A method according to claim 18, wherein the step of washing the pellets comprises:
washing the pellets by suspending the pellets in normal saline to form a mixture;
vortexing the mixture; and
centrifuging the mixture to isolate residual carbonaceous particles in the form of pellets.

20. A method according to claim 18, wherein the step of drying the washed pellets comprises transferring sufficient amount of pellets to a quartz filter such that the total mass of the elemental carbon particles in the pellets on the filter is between about 1 μg and 100 μg.

21. A method according to claim 12, wherein the step of analyzing the elemental carbon comprises measuring the concentration of elemental carbon in the washed pellets using a carbon analyzer.

22. A method of determining the amount of carbonaceous particles in biological sample comprising the steps of:
weighing the biological sample;
solubilizing the biological sample in a solvent to separate carbonaceous particles including elemental carbon from the biological sample; and
isolating, by high speed centrifugation, residual carbonaceous particles in the form of pellets.
washing the pellets to remove impurities;
drying the pellets; and
analyzing the amounts of organic and elemental carbon in the pellets using a carbon analyzer.

23. A method according to claim 22, wherein the biological sample is selected from a group consisting of lung tissues, lung epithelial cells, and macrophages.

24. A method according to claim 22, wherein the biological sample comprises carbonaceous particles selected from a group consisting of Diesel Exhaust Particles, Ultra-fine Carbon Black, Nano-diamonds, and Carbon-nanotubes.
